(19) United States
(12) Patent Application Publication
(10) Pub. No.: US 2014/0127330 A1
(43) Pub. Date: May 8, 2014

(54) METHOD OF MIXING GASES INCLUDING NITRIC OXIDE

(71) Applicant: GENO LLC, Cocoa, FL (US)

(72) Inventors: David H. Fine, Cocoa Beach, FL (US); Edward Bromberg, Orlando, FL (US); Lucas Gamero, Oviedo, FL (US); Ryan Denton, Titusville, FL (US); Gregory Vasquez, Cocoa, FL (US); Bryan Johnson, Orlando, FL (US)

(21) Appl. No.: 14/071,841

(22) Filed: Nov. 5, 2013

Related U.S. Application Data
(60) Provisional application No. 61/722,621, filed on Nov. 5, 2012.

(51) Int. Cl. A61K 33/00

(52) U.S. Cl. CPC A61K 33/00
USPC 424/718

(57) ABSTRACT

A method of delivering nitric oxide can include mixing a first gas including oxygen and a second gas including a nitric oxide-releasing agent within a receptacle to form a gas mixture, where the receptacle includes an inlet, an outlet and a reducing agent, contacting the nitric oxide-releasing agent in the gas mixture with the reducing agent to generate nitric oxide, and delivering the gas mixture including nitric oxide from the receptacle to a mammal.

### CLAIM OF PRIORITY

This application claims the benefit of prior U.S. Provisional Application No. 61/722,621 filed on Nov. 5, 2012, which is incorporated by reference in its entirety.

### TECHNICAL FIELD

The invention relates to mixing a gas flow including oxygen and a gas flow including a nitric oxide-releasing agent within a receptacle, which converts the nitric oxide-releasing agent to nitric oxide.

### BACKGROUND

Nitric oxide (NO), also known as nitrosyl radical, is a free radical that is an important signalling molecule. For example, NO can cause smooth muscles in blood vessels to relax, thereby resulting in vasodilation and increased blood flow through the blood vessel. These effects can be limited to small biological regions since NO can be highly reactive with a lifetime of a few seconds and can be quickly metabolized in the body.

Some disorders or physiological conditions can be mediated by inhalation of nitric oxide. The use of low concentrations of inhaled nitric oxide can prevent, reverse, or limit the progression of disorders which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism and idiopathic or primary pulmonary hypertension or chronic hypoxia.

Generally, nitric oxide can be inhaled or otherwise delivered to the individual's lungs. Providing a therapeutic dose of NO could treat a patient suffering from a disorder or physiological condition that can be mediated by inhalation of NO or supplement or minimize the need for traditional treatments in such disorders or physiological conditions. Typically, the NO gas can be supplied in a bottled gaseous form diluted in nitrogen gas (N₂). Great care should be taken to prevent the presence of even trace amounts of oxygen (O₂) in the tank of NO gas because the NO, in the presence of O₂, can be oxidized to nitrogen dioxide (NO₂). Unlike NO, the part per million levels of NO₂ gas can be highly toxic if inhaled and can form nitric and nitrous acid in the lungs.

### SUMMARY

In one aspect, a method of delivering nitric oxide can include mixing a first gas and a second gas to form a gas mixture.

In some embodiments, a first gas can include oxygen. For example, a first gas can be air or oxygen-enriched air. A first gas can also be an oxygen-enriched gas, in other words, a gas in which oxygen has been added.

In some embodiments, a method can include communicating a first gas through a gas conduit to the receptacle. In some cases, a first gas can be continuously communicated through the gas conduit. In other cases, a first gas can be intermittently communicated through the gas conduit. In some embodiments, communicating a first gas through a gas conduit to the receptacle can include communicating the first gas through the gas conduit in one or more pulses. Communicating a first gas through a gas conduit can be performed using a ventilator.

In some embodiments, a second gas can include a nitric oxide-releasing agent. A nitric oxide-releasing agent can include one or more of nitric oxide (NO), nitrogen dioxide (NO₂), dinitrogen tetroxide (N₂O₄) or nitrite ions (NO₂). Nitrite ions can be introduced in the form of a nitrite salt, such as sodium nitrite. In some embodiments, a second gas can include an inert gas (e.g. N₂). In other embodiments, a second gas can include oxygen or air.

In some embodiments, a method can include supplying a second gas into the gas conduit. In some cases, the second gas can be supplied into the gas conduit immediately prior to the receptacle or at the receptacle.

In some embodiments, mixing a first gas and a second gas can occur within a receptacle to form a gas mixture. In some embodiments, a receptacle can include an inlet, an outlet and a reducing agent. A reducing agent can include one or more compounds capable of donating an electron to another species during a reduction-oxidation (redox) reaction. A reducing agent can include hydroquinone, glutathione, and/or one or more reduced metal salts such as Fe(II), Mo(VI), NaI, Ti(III) or Cr(III), thiols, or NO₂⁻. A reducing agent can also include one or more of 3,4 dihydroxy-cyclobutene-dione, maleic acid, croconic acid, dihydroxy-fumaric acid, tetra-hydroxy-quinone, p-toluene-sulfonic acid, tricholor-acetic acid, mandelic acid, 2-fluoro-mandelic acid, or 2,3,5,6-tetrafluoro-mandelic acid.

In some embodiments, a reducing agent can be an antioxidant. An antioxidant can include any number of common antioxidants, including ascorbic acid, alpha tocopherol, and/or gamma tocopherol. A reducing agent can include a salt, ester, anhydride, crystalline form, or amorphous form of any of the reducing agents listed above.

In some embodiments, a receptacle can include a support. A support can be any material that has at least one solid or non-fluid surface (e.g. a gel). It can be advantageous to have a support that has at least one surface with a large surface area. In preferred embodiments, the support can be porous. One example of a support can be surface-active material, for example, a material with a large surface area that is capable of retaining water or absorbing moisture. Specific examples of surface active materials can include silica gel or cotton.

In some embodiments, the concentration of nitric oxide in a gas mixture can be at least 0.01 ppm, at least 0.05 ppm, at least 0.1 ppm, at least 0.5 ppm, at least 1 ppm, at least 1.5 ppm, at least 2 ppm or at least 5 ppm. The concentration of nitric oxide in a gas mixture can be at most 100 ppm, at most 80 ppm, at most 60 ppm, at most 40 ppm, at most 25 ppm, at most 20 ppm, at most 10 ppm, at most 5 ppm or at most 2 ppm. Preferably, the concentration of nitric oxide in a gas mixture can be at least 0.01 ppm and at most 40 ppm, at least 0.01 ppm and at most 25 ppm, or at least 0.01 ppm and at most 2 ppm.

In some embodiments, the concentration of nitrogen dioxide in the gas mixture delivered to the mammal can be less than 1 ppm, less than 0.5 ppm, less than 0.2 ppm, less than 0.1 ppm or less than 0.05 ppm.

In some embodiments, a method can include contacting the nitric oxide-releasing agent in the gas mixture with the reducing agent to generate nitric oxide.

In some embodiments, a method can include delivering the gas mixture including nitric oxide from a receptacle to a mammal. In some instances, the mammal can be a human.

In some embodiments, delivering the gas mixture including nitric oxide from the receptacle to the mammal can include passing the gas mixture through a delivery conduit located between the receptacle and a patient interface. A patient interface can include a mouth piece, nasal cannula, face mask, fully-sealed face mask or an endotracheal tube. A patient interface can be coupled to a delivery conduit. A delivery conduit can include a ventilator or an anesthesia machine.

In some embodiments, the volume of the receptacle can be greater than the volume of the delivery conduit. The volume of the receptacle can be at least 1.5 times, at least 3 times, at least 4 times or at least 5 times the volume of the delivery conduit.

In some embodiments, delivering the gas mixture including nitric oxide from the receptacle to the mammal can include continuously providing the nitric oxide to the mammal. In other embodiments, delivering the gas mixture including nitric oxide from the receptacle to the mammal can include intermittently providing the gas mixture to the mammal.

In some embodiments, delivering the gas mixture including nitric oxide from the receptacle to the mammal can include pulsing the gas mixture. In some embodiments, pulsing can include providing the gas mixture for one or more pulses. In some embodiments, pulsing can include providing the gas mixture for two or more pulses. A pulse can last for a few seconds up to as long as several minutes. In one embodiment, a pulse can last for 1, 5, 10, 15, 20, 25, 30, 35, 40, 45, 50, 55 or 60 seconds. In another embodiment, a pulse can last for 1, 2, 3, 4 or 5 minutes. In a preferred embodiment, a pulse can last for 0.5-10 seconds, most preferably 1-6 seconds.

In some embodiments, delivering the gas mixture including nitric oxide from the receptacle to the mammal can include providing the gas mixture to the mammal in a pre-determined delivery sequence of one or more pulses. For example, a pulse can be delivered at a predetermined interval and for a predetermined duration.

In some embodiments, the volume of the receptacle can be greater than the volume of the gas mixture in a pulse. The volume of the receptacle can be at least 1.5 times, at least 3 times, at least 4 times or at least 5 times the volume of the gas mixture in a pulse.

In some embodiments, a gas mixture can be stored in a receptacle. In some embodiments, a gas mixture can be stored in a receptacle during or between pulses. In some instances, storing the gas mixture in the receptacle can be for a predetermined period of time, which can be at least 1 second, at least 2 seconds, at least 6 seconds, at least 10 seconds, at least 20 seconds, at least 30 seconds or at least 1 minute.

In some embodiments, the concentration of nitric oxide in each pulse can vary by less than 10%, by less than 5%, or by less than 2%. In some embodiments, using intermittent delivery, the concentration of nitric oxide in each pulse or on-period can vary by less than 10 ppm, less than 5 ppm, less than 2 ppm or less than 1 ppm.

Other features, objects, and advantages will be apparent from the description, drawings, and claims.

### BRIEF DESCRIPTION OF THE DRAWINGS

FIG. 1 is an illustration of a receptacle.

FIG. 2 is an illustration of a system including a receptacle.

FIG. 3 is a drawing depicting a system including a receptacle.

FIG. 4 is a graph showing nitric oxide and nitrogen dioxide concentrations as a function of time in comparison to a ventilator flow rate.

FIG. 5 is a graph showing nitric oxide and nitrogen dioxide concentrations as a function of time in comparison to a ventilator flow rate.

FIG. 6 is a graph showing nitric oxide concentration as a function of time in comparison to a ventilator flow rate.

FIG. 7 is a graph showing nitric oxide concentration as a function of time in comparison to a ventilator flow rate.

FIG. 8 is a graph showing nitric oxide concentration as a function of time in comparison to a ventilator flow rate.

FIG. 9 is a graph showing nitric oxide concentration as a function of time in comparison to a ventilator flow rate.

### DETAILED DESCRIPTION

Nitric oxide, also known as nitrosyl radical, is a free radical that is an important signaling molecule in pulmonary vessels. Nitric oxide can moderate pulmonary hypertension caused by elevation of the pulmonary arterial pressure. Inhaling low concentrations of nitric oxide, for example, in the range of 0.01-100 ppm can rapidly and safely decrease pulmonary hypertension in a mammal by vasodilation of pulmonary vessels.

Some disorders or physiological conditions can be mediated by inhalation of nitric oxide. The use of low concentrations of inhaled nitric oxide can prevent, reverse, or limit the progression of disorders which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism and idiopathic or primary pulmonary hypertension or chronic hypoxia. Advantageously, nitric oxide can be generated and delivered in the absence of harmful side products, such as nitrogen dioxide. The nitric oxide can be generated at a concentration suitable for delivery to a mammal in need of treatment.

When delivering nitric oxide (NO) for therapeutic use to a mammal, it can be important to avoid delivery of nitrogen dioxide (NO₂) to the mammal. Nitrogen dioxide (NO₂) can be formed by the oxidation of nitric oxide (NO) with oxygen (O₂). The rate of formation of nitrogen dioxide (NO₂) can be proportional to the oxygen (O₂) concentration multiplied by the square of the nitric oxide (NO) concentration. A NO delivery system can convert nitrogen dioxide (NO₂) to nitric oxide (NO). Additionally, nitric oxide can form nitrogen dioxide at increased concentrations.

A nitric oxide delivery system can include a receptacle. A receptacle can include an inlet and an outlet. A receptacle can convert a nitric oxide-releasing agent to nitric oxide (NO). A nitric oxide-releasing agent can include one or more of nitrogen dioxide (NO₂), dinitrogen tetroxide (N₂O₄) or nitrite ions (NO₂⁻). Nitrite ions can be introduced in the form of a nitrite salt, such as sodium nitrite.

A receptacle can include a reducing agent or a combination of reducing agents. A number of reducing agents can be used depending on the activities and properties as determined by a person of skill in the art. In some embodiments, a reducing agent can include a hydroquinone, glutathione, and/or one or more reduced metal salts such as Fe(II), Mo(VI), NaI, Ti(III) or Cr(III), thiols, or NO₂. A reducing agent can include 3,4 dihydroxy-cyclobutene-dione, maleic acid, croconic acid, dihydroxy-fumaric acid, tetra-hydroxy-quinone, p-toluene-sulfonic acid, tricholor-acetic acid, mandelic acid, 2-fluoro-mandelic acid, or 2,3,5,6-tetrafluoro-mandelic acid. A reducing agent can be safe (i.e., non-toxic and/or non-caustic) for inhalation by a mammal, for example, a human. A reducing agent can be an antioxidant. An antioxidant can include any number of common antioxidants, including ascorbic acid, alpha tocopherol, and/or gamma tocopherol. A reducing agent can include a salt, ester, anhydride, crystalline form, or amorphous form of any of the reducing agents listed above. A reducing agent can be used dry or wet. For example, a reducing agent can be in solution. A reducing agent can be at different concentrations in a solution. Solutions of the reducing agent can be saturated or unsaturated. While a reducing agent in organic solutions can be used, a reducing agent in an aqueous solution is preferred. A solution including a reducing agent and an alcohol (e.g. methanol, ethanol, propanol, isopropanol, etc.) can also be used.

A receptacle can include a support. A support can be any material that has at least one solid or non-fluid surface (e.g. a gel). It can be advantageous to have a support that has at least one surface with a large surface area. In preferred embodiments, the support can be porous or permeable. One example of a support can be surface-active material, for example, a material with a large surface area that is capable of retaining water or absorbing moisture. Specific examples of surface active materials can include silica gel or cotton. The term “surface-active material” denotes that the material supports an active agent on its surface.

A support can include a reducing agent. Said another way, a reducing agent can be part of a support. For example, a reducing agent can be present on a surface of a support. One way this can be achieved can be to coat a support, at least in part, with a reducing agent. In some cases, a system can be coated with a solution including a reducing agent. Preferably, a system can employ a surface-active material coated with an aqueous solution of antioxidant as a simple and effective mechanism for making the conversion. Generation of NO from a nitric oxide-releasing agent performed using a support with a reducing agent can be the most effective method, but a reducing agent alone can also be used to convert nitric oxide-releasing agent to NO.

In some circumstances, a support can be a matrix or a polymer, more specifically, a hydrophilic polymer. A support can be mixed with a solution of the reducing agent. The solution of reducing agent can be stirred and strained with the support and then drained. The moist support-reducing agent mixture can be dried to obtain the proper level of moisture. Following drying, the support-reducing agent mixture may still be moist or may be dried completely. Drying can occur using a heating device, for example, an oven or autoclave, or can occur by air drying.

In general, a nitric oxide-releasing agent can be converted to NO by bringing a gas including the nitric oxide-releasing agent in contact with a reducing agent. In one example, a gas including a nitric oxide-releasing agent can be passed over or through a support including a reducing agent. When the reducing agent is ascorbic acid (i.e. vitamin C), the conversion of nitrogen dioxide to nitric oxide can be quantitative at ambient temperatures.

The generated nitric oxide can be delivered to a mammal, which can be a human. To facilitate delivery of the nitric oxide, a system can include a patient interface. Examples of a patient interface can include a mouth piece, nasal cannula, face mask, fully-sealed face mask or an endotracheal tube. A patient interface can be coupled to a delivery conduit. A delivery conduit can include a ventilator or an anesthesia machine.

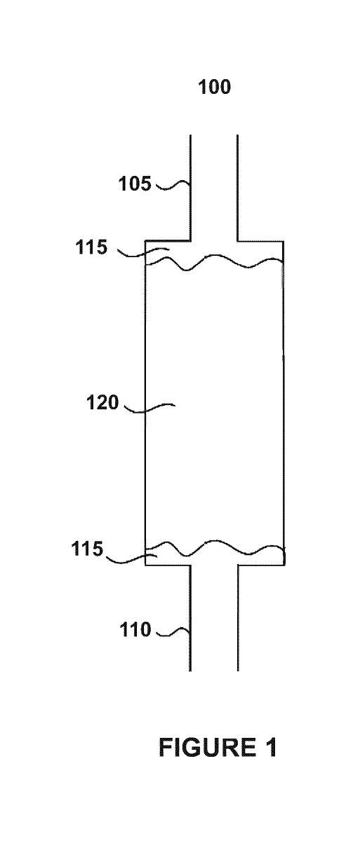

FIG. 1 illustrates one embodiment of a receptacle for generating NO by converting a nitric oxide-releasing agent to NO. The receptacle 100 can include an inlet 105 and an outlet 110. An example of a receptacle can be a cartridge. A cartridge can be inserted into and removed from an apparatus, platform or system. Preferably, a cartridge is replaceable in the apparatus, platform or system, and more preferably, a cartridge can be disposable. Screen and glass wool 115 can be located at either or both of the inlet 105 and the outlet 110. The remainder of the receptacle 100 can include a support. In a preferred embodiment, a receptacle 100 can be filled with a surface-active material 120. The surface-active material 120 can be soaked with a saturated solution of antioxidant in water to coat the surface-active material. The screen and glass wool 115 can also be soaked with the saturated solution of antioxidant in water before being inserted into the receptacle 100.

In general, a process for converting a nitric oxide-releasing agent to NO can include passing a gas including a nitric oxide-releasing agent into the inlet 105. The gas can be communicated to the outlet 110 and into contact with a reducing agent. In a preferred embodiment, the gas can be fluidly communicated to the outlet 110 through the surface-active material 120 coated with a reducing agent. As long as the surface-active material remains moist and the reducing agent has not been used up in the conversion, the general process can be effective at converting a nitric oxide-releasing agent to NO at ambient temperature.

The inlet 105 may receive the gas including a nitric oxide-releasing agent from a gas pump that fluidly communicates the gas over a diffusion tube or a permeation cell. The inlet 105 also may receive the gas including a nitric oxide-releasing agent, for example, from a pressurized bottle of a nitric oxide-releasing agent. A pressurized bottle may also be referred to as a tank. The inlet 105 also may receive a gas including a nitric oxide-releasing agent can be NO₂ gas in nitrogen (N₂), air, or oxygen (O₂). A wide variety of flow rates and NO₂ concentrations have been successfully tested, ranging from only a few ml per minute to flow rates of up to 5,000 ml per minute.

The conversion of a nitric oxide-releasing agent to NO can occur over a wide range of concentrations of a nitric oxide-releasing agent. For example, experiments have been carried out at concentrations in air of from about 2 ppm NO₂ to 100 ppm NO₂, and even to over 1000 ppm NO₂. In one example, a receptacle that was approximately 6 inches long and had a diameter of 1.5-inches was packed with silica gel that had first been soaked in a saturated aqueous solution of ascorbic acid. The moist silica gel was prepared using ascorbic acid designated as A.C.S reagent grade 99.1% pure from Aldrich Chemical Company and silica gel from Fischer Scientific International, Inc., designated as S8 32-1, 40 of Grade of 35 to 70 sized mesh. Other sizes of silica gel can also be effective. For example, silica gel having an eighth-inch diameter can also work.

In another example, silica gel was moistened with a saturated solution of ascorbic acid that had been prepared by mixing 35% by weight ascorbic acid in water, stiffing, and straining the water/ascorbic acid mixture through the silica gel, followed by draining. The conversion of NO₂ to NO can proceed well when the support including the reducing agent, for example, silica gel coated with ascorbic acid, is moist. In a specific example, a receptacle filled with the wet silica gel/ascorbic acid was able to convert 1000 ppm of NO₂ in air to NO at a flow rate of 150 ml per minute, quantitatively, non-stop for over 12 days.

A receptacle can be used for inhalation therapy. In addition to converting a nitric oxide-releasing agent to nitric oxide to be delivered during inhalation therapy, a receptacle can remove any NO₂ that chemically forms during inhalation therapy (e.g., nitric oxide that is oxidized to form nitrogen dioxide). In one such example, a receptacle can be used as a NO₂ scrubber for NO inhalation therapy that delivers NO from a pressurized bottle source. A receptacle may be used to help ensure that no harmful levels of NO₂ are inadvertently inhaled by the patient.

In addition, a receptacle may be used to supplement or replace some or all of the safety devices used during inhalation therapy in conventional NO inhalation therapy. For example, one type of safety device can warn of the presence of NO₂ in a gas when the concentration of NO₂ exceeds a preset or predetermined limit, usually 1 part per million or greater of NO₂. Such a safety device may be unnecessary when a receptacle is positioned in a NO delivery system just prior to the patient breathing the NO laden gas. A receptacle can convert any NO₂ to NO just prior to the patient breathing the NO laden gas, making a device to warn of the presence of NO₂ in gas unnecessary.

Furthermore, a receptacle placed near the exit of inhalation equipment, gas lines or gas tubing can also reduce or eliminate problems associated with formation of NO₂ that occur due to transit times in the equipment, lines or tubing. As such, use of a receptacle can reduce or eliminate the need to ensure the rapid transit of the gas through the gas plumbing lines that is needed in conventional applications. Also, a receptacle can allow the NO gas to be used with gas balloons to control the total gas flow to the patient.

Alternatively or additionally, a NO₂ removal receptacle can be inserted just before the attachment of the delivery system to the patient to further enhance safety and help ensure that all traces of the toxic NO₂ have been removed. The NO₂ removal receptacle may be a receptacle used to remove any trace amounts of NO₂. Alternatively, the NO₂ removal receptacle can include heat-activated alumina. A receptacle with heat-activated alumina, such as supplied by Fisher Scientific International, Inc., designated as ASOS-212, of 8-14 sized mesh can be effective at removing low levels of NO₂ from an air or oxygen stream, and yet, can allow NO gas to pass through without loss. Activated alumina, and other high surface area materials like it, can be used to scrub NO₂ from a NO inhalation line.

In another example, a receptacle can be used to generate NO for therapeutic gas delivery. Because of the effectiveness of a receptacle in converting nitric oxide-releasing agents to NO, nitrogen dioxide (gaseous or liquid) or dinitrogen tetroxide can be used as the source of the NO. When nitrogen dioxide or dinitrogen tetroxide is used as a source for generation of NO, there may be no need for a pressurized gas bottle to provide NO gas to the delivery system. By eliminating the need for a pressurized gas bottle to provide NO, the delivery system may be simplified as compared with a conventional apparatus that is used to deliver NO gas to a patient from a pressurized gas bottle of NO gas. A NO delivery system that does not use pressurized gas bottles may be more portable than conventional systems that rely on pressurized gas bottles.

In some delivery systems, the amount of nitric oxide-releasing agent in a gas can be approximately equivalent to the amount of nitric oxide to be delivered to a patient. For example, if a therapeutic dose of 20 ppm of nitric oxide is to be delivered to a patient, a gas including 20 ppm of a nitric oxide-releasing agent (e.g., NO₂) can be released from a gas bottle or a diffusion tube. The gas including 20 ppm of a nitric oxide-releasing agent can be passed through one or more receptacles to convert the 20 ppm of nitric oxide-releasing agent to 20 ppm of nitric oxide for delivery to the patient. However, in other delivery systems, the amount of nitric oxide-releasing agent in a gas can be greater than the amount of nitric oxide to be delivered to a patient. For example, a gas including 800 ppm of a nitric oxide-releasing agent can be released from a gas bottle or a diffusion tube. The gas including 800 ppm of a nitric oxide-releasing agent can be passed through one or more receptacles to convert the 800 ppm of nitric oxide-releasing agent to 800 ppm of nitric oxide. The gas including 800 ppm of nitric oxide can then be diluted in a gas including oxygen (e.g., air) to obtain a gas mixture with 20 ppm of nitric oxide for delivery to a patient. Traditionally, the mixing of a gas including nitric oxide with a gas including oxygen to dilute the concentration of nitric oxide has occurred in a line or tube of the delivery system. The mixing of a gas including nitric oxide with a gas including oxygen can cause problems because nitrogen dioxide can form. To avoid this problem, two approaches have been used. First, the mixing of the gases can be performed in a line or tube immediately prior to the patient interface, to minimize the time nitric oxide is exposed to oxygen, and consequently, reduce the nitrogen dioxide formation. Second, a receptacle can be placed at a position downstream of the point in the line or tubing where the mixing of the gases occurs, in order to convert any nitrogen dioxide formed back to nitric oxide.

While these approaches can minimize the nitrogen dioxide levels in a gas delivered to a patient, these approaches have some drawbacks. Significantly, both of these approaches mix a gas including nitric oxide with a gas including oxygen in a line or tubing of the system. One problem can be that lines and tubing in a gas delivery system can have a limited volume, which can constrain the level of mixing. Further, a gas in lines and tubing of a gas delivery system can experience variations in pressure and flow rates. Variations in pressure and flow rates can lead to an unequal distribution of the amount each gas in a mixture throughout a delivery system. Moreover, variations in pressure and flow rates can lead to variations in the amount of time nitric oxide is exposed to oxygen within a gas mixture. One notable example of this arises with the use of a ventilator, which pulses gas through a delivery system. Because of the variations in pressure, variations in flow rates and/or the limited volume of the lines or tubing where the gases are mixed, a mixture of the gases can be inconsistent, leading to variation in the amount of nitric oxide, nitrogen dioxide, nitric oxide-releasing agent and/or oxygen between any two points in a delivery system.

To address these problems, a receptacle can also be used to mix a first gas and a second gas. A first gas can include oxygen; more specifically, a first gas can be air. A second gas can include a nitric oxide-releasing agent and/or nitric oxide. A first gas and a second gas can be mixed within a receptacle to form a gas mixture. The mixing can be an active mixing performed by a mixer within a receptacle. For example, a mixer can be a moving support. The mixing within a receptacle can also be a passive mixing, for example, the result of diffusion.

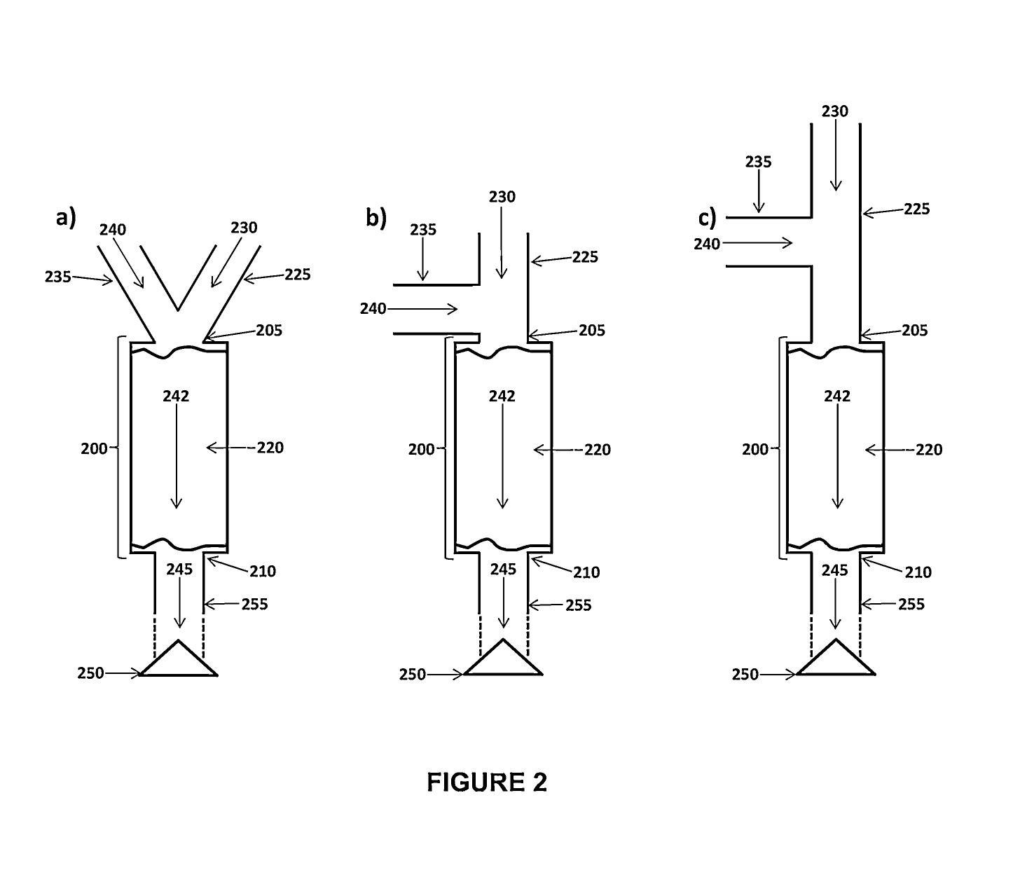

As shown in FIGS. 2a, 2b and 2c, a receptacle 200 can be coupled to a gas conduit 225. A first gas 230 including oxygen can be communicated through a gas conduit 225 to the receptacle 200. The communication of the first gas through the gas conduit can be continuous or it can be intermittent. For instance, communicating the first gas intermittently can include communicating the first gas through the gas conduit in one or more pulses. Intermittent communication of the first gas through gas conduit can be performed using a gas bag, a pump, a hand pump, an anesthesia machine or a ventilator.

A gas conduit can include a gas source. A gas source can include a gas bottle, a gas tank, a permeation cell or a diffusion tube. Nitric oxide delivery systems including a gas bottle, a gas tank a permeation cell or a diffusion tube are described, for example, in U.S. Pat. Nos. 7,560,076 and 7,618,594, each of which are incorporated by reference in its entirety. Alternatively, a gas source can include a reservoir and restrictor, as described in U.S. patent application Ser. Nos. 12/951,811, 13/017,768 and 13/094,535, each of which is incorporated by reference in its entirety. A gas source can include a pressure vessel, as described in U.S. patent application Ser. No. 13/492,154, which is incorporated by reference in its entirety. A gas conduit can also include one or more additional receptacles. Additional components including one or more sensors for detecting nitric oxide levels, one or more sensors for detecting nitrogen dioxide levels, one or more sensor for detecting oxygen levels, one or more humidifiers, valves, tubing or lines, a pressure regulator, flow regulator, a calibration system and/or filters can also be included in a gas conduit.

A second gas 240 can also be communicated to a receptacle 200. A second gas can be supplied into a gas conduit, as shown in FIGS. 2b and 2c. Preferably, a second gas 240 can be supplied into a gas conduit 225 immediately prior to a receptacle 200, as shown in FIG. 2b. A second gas 240 can be supplied into a gas conduit 225 via a second gas conduit 235, which can join or be coupled to the gas conduit 225. Once a second gas 240 is supplied into a gas conduit 225, both the first gas 230 and the second gas 240 can be communicated in the inlet 205 of a receptacle 200 for mixing. Alternatively, a second gas 240 can be supplied at a receptacle 200, as show in FIG. 2a. For example, a second gas 240 can be supplied directly into the inlet 205 of a receptacle 200.

Once a first gas 230 and a second gas 240 are within a receptacle 200, a first gas 230 and a second gas 240 can mix to form a gas mixture 242 including oxygen and one or more of nitric oxide, a nitric oxide-releasing agent (which can be nitrogen dioxide) and nitrogen dioxide. The gas mixture 242 can contact a reducing agent, which can be on a support 220 within the receptacle. The reducing agent can convert nitric oxide-releasing agent and/or nitrogen dioxide in the gas mixture to nitric oxide.

The gas mixture including nitric oxide 245 can then be delivered to a mammal, most preferably, a human patient. The concentration of nitric oxide in a gas mixture can be at least 0.01 ppm, at least 0.05 ppm, at least 0.1 ppm, at least 0.5 ppm, at least 1 ppm, at least 1.5 ppm, at least 2 ppm or at least 5 ppm. The concentration of nitric oxide in a gas mixture can be at most 100 ppm, at most 80 ppm, at most 60 ppm, at most 40 ppm, at most 25 ppm, at most 20 ppm, at most 10 ppm, at most 5 ppm or at most 2 ppm.

Delivering the gas mixture including nitric oxide from the receptacle 200 to the mammal can include passing the gas mixture through a delivery conduit. A delivery conduit 255 can be located between the receptacle 200 and a patient interface 250. In some embodiments, a delivery conduit 255 can be coupled to the outlet 210 of a receptacle 200 and/or coupled to the patient interface 250. As indicated by the dashed lines in FIGS. 2a, 2b and 2c, a delivery conduit can include additional components, for example, a humidifier or one or more additional receptacles.

Delivery of a gas mixture can include continuously providing the gas mixture to the mammal. When the delivery of the gas mixture includes continuously providing the gas mixture to the mammal, the volume of the receptacle can be greater than the volume of the delivery conduit. The larger volume of the receptacle can help to ensure that the gas mixture is being thoroughly mixed prior to delivery. Generally, more complete mixing can occur as the ratio of the volume of the receptacle to the volume of the delivery conduit increases. A preferable level of mixing can occur when the volume of the receptacle is at least twice the volume of the delivery conduit. The volume of the receptacle can also be at least 1.5 times, at least 3 times, at least 4 times or at least 5 times the volume of the delivery conduit.

When the volume of the receptacle is greater than the volume of the delivery conduit or the volume of gas mixture in the delivery conduit, the gas mixture may not go directly from the receptacle to the mammal, but instead, can be delayed in the receptacle or delivery conduit. It is this delay that can provide the time needed to mix the gas so that the NO concentration remains constant within a breath.

This delay can result in the storage of the gas mixture in the receptacle. The gas mixture can be stored in the receptacle for a predetermined period of time. The predetermined period of time can be at least 1 second, at least 2 seconds, at least 6 seconds, at least 10 seconds, at least 20 seconds, at least 30 seconds or at least 1 minute.

The mixing that occurs due to the delay of the gas mixture (i.e. storage of the gas mixture in a receptacle) can be so effective that the intra-breath variation can be identical to what could be achieved under ideal conditions when premixed gas was provided. This can be referred to as “perfect mixing.” For continuous delivery, this can mean that the concentration of nitric oxide in the gas mixture delivered to a mammal remains constant over a period of time (e.g. at least 1 min, at least 2 min, at least 5 min, at least 10 min or at least 30 min). For a concentration to remain constant, the concentration can remain with a range of at most ±10%, at most ±5%, or at most ±2% of a desired concentration for delivery.

Delivery of the gas mixture can include intermittently providing the gas mixture to the mammal. Intermittent delivery of a gas mixture can be the result of intermittent communication of a first or second gas into the system. Said another way, intermittent communication of a first or second gas through a gas conduit can result in an increased area of pressure, which can traverse into the receptacle causing intermittent communication of the gas mixture. Intermittent delivery can be performed using a gas bag, a pump, a hand pump, an anesthesia machine or a ventilator.

The intermittent delivery can include an on-period, when the gas mixture is delivered to a patient, and an off-period, when the gas mixture is not delivered to a patient. Intermittent delivery can include delivering one or more pules of the gas mixture.

An on-period or a pulse can last for a few seconds up to as long as several minutes. In one embodiment, an on-period or a pulse can last for 1, 5, 10, 15, 20, 25, 30, 35, 40, 45, 50, 55 or 60 seconds. In another embodiment, the on-period or a pulse can last for 1, 2, 3, 4 or 5 minutes. In a preferred embodiment, an on-period or a pulse can last for 0.5-10 seconds, most preferably 1-6 seconds.

Intermittent delivery can include a plurality of on-periods or pulses. For example, intermittent delivery can include at least 1, at least 2, at least 5, at least 10, at least 50, at least 100 or at least 1000 on-periods or pulses.

The timing and duration of each on-period or pulse of the gas mixture can be pre-determined. Said another way, the gas mixture can be delivered to a patient in a pre-determined delivery sequence of one or more on-periods or pulses. This can be achieved using an anesthesia machine or a ventilator, for example.

When the delivery of the gas mixture includes intermittently providing the gas mixture to the mammal, the volume of the receptacle can be greater than the volume of the gas mixture in a pulse or on-period. The larger volume of the receptacle can help to ensure that the gas mixture is being thoroughly mixed prior to delivery. Generally, more complete mixing can occur as the ratio of the volume of the receptacle to the volume of the gas mixture in a pulse or on-period delivered to a mammal increases. A preferable level of mixing can occur when the volume of the receptacle is at least twice the volume of the gas mixture in a pulse or on-period. The volume of the receptacle can also be at least 1.5 times, at least 3 times, at least 4 times or at least 5 times the volume of the gas mixture in a pulse or on-period.

When the volume of the receptacle is greater than the volume of the volume of the gas mixture in a pulse or on-period, the gas mixture may not go directly from the receptacle to the mammal, but instead, can be delayed in the receptacle or delivery conduit for one or more pulses or on-periods. It is this delay that can provide the time needed to mix the gas so that the NO concentration remains constant between delivered pulses or on-periods.

In addition to storage as a result of off-periods, the delay caused by the differing volumes can result in the storage of the gas mixture in the receptacle. The gas mixture can be stored in the receptacle for a predetermined period of time. The predetermined period of time can be during or between pulses or on-periods. The predetermined period of time can be at least 1 second, at least 2 seconds, at least 6 seconds, at least 10 seconds, at least 20 seconds, at least 30 seconds or at least 1 minute.

The mixing that occurs due to the delay of the gas mixture (i.e. storage of the gas mixture in a receptacle) can be so effective that the intra-breath variation can be identical to what could be achieved under ideal conditions when premixed gas was provided. Intermittent delivery an include providing the gas mixture for two or more pulses or on-periods. Using intermittent delivery, the concentration of nitric oxide in each pulse or on-period can vary by less than 10%, by less than 5%, or by less than 2%. In other words, the variation between the concentration of nitric oxide in a first pulse and the concentration of nitric oxide in a second pulse is less than 10% (or less than 5% or 2%) of the concentration of nitric oxide in the first pulse. In another embodiment, using intermittent delivery, the concentration of nitric oxide in each pulse or on-period can vary by less than 10 ppm, less than 5 ppm, less than 2 ppm or less than 1 ppm. Said another way, the difference between the concentration of nitric oxide in a first pulse and the concentration of nitric oxide in a second pulse is less than 10 ppm, less than 5 ppm, less than 2 ppm or less than 1 ppm.

### Examples

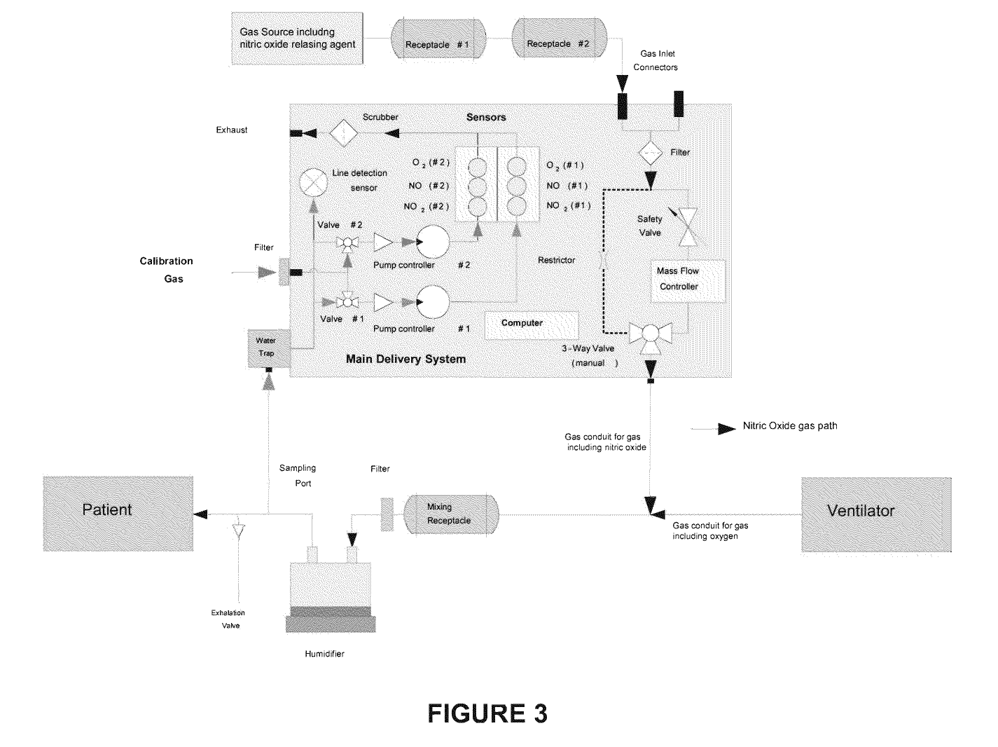

FIG. 3 shows the flow path schematics of an embodiment of a system where a receptacle is used for mixing gas. In this configuration, the gas source including a nitric oxide-releasing agent can be NO₂ in air, for example a bottle of 800 ppm NO₂ in air. Alternatively, the gas source can also be from a liquid source. If a liquid source is used, then the concentration of the source can be variable. In some instances, the concentration of NO₂ can be from about 1000 ppm down to about 50 ppm. The concentration of NO₂ from a liquid source can be controlled by controlling the temperature of the source.

The embodiment shown in FIG. 3 has demonstrated the ability to supply a constant concentration of NO for the duration of the inspired breath. The functions of a receptacle, shown as a mixing receptacle in FIG. 3, can include:

1) To convert any NO₂ that may have formed in the line into NO.

2) To provide adequate mixing of NO in the patient circuit prior to inhalation.

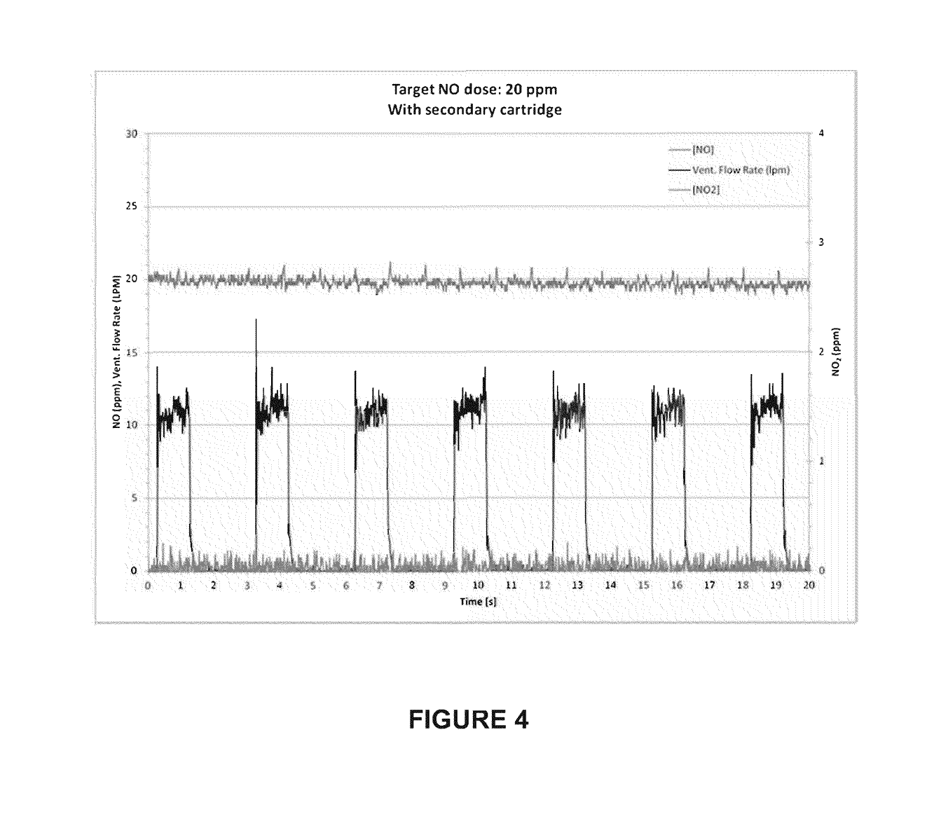

FIG. 4 shows a typical response of a system as embodied in FIG. 3 configured to deliver 20 ppm of NO. The NO₂ values (bottom) are shown (right hand axis). These measurements were obtained using the electrochemical gas analyzers that are part of the system. It is to be noted that the NO₂ levels can be essentially zero when the NO level is at 20 ppm. As shown by the middle plot, the ventilator flow rate is shown (left hand axis). To focus on the worst case scenario, the bias flow of the ventilator was set to zero.

The system was delivering 20 ppm of NO in 21% oxygen using an infant ventilator (Bio-Med Devices CV2+) with the ventilator settings shown in Table 1. The slower breathing rate was used as the worst case for NO mixing, because of the longer pause during exhalation.

TABLE 1
Ventilator Settings
Ventilator Settings

| | Pressure |
|---|---|
| Mode | Control |
| Rate (BPM) | 40 |
| Inspiratory Time INSP (sec) | 0.50 |
| Flow (LPM) | 6.0 |
| I:E Ratio | 1:2.0 |

The NO measurements were within product specifications (±20%). The conversion of NO₂ to NO in the receptacle overcomes the formation of NO₂ that is caused by the delay due to mixing.

As discussed above, the mixing can occur if the volume of the receptacle exceeds the ventilator pulse volume. For example, a 6000 ml/min and 40 breaths per minute the volume of the pulse is 150 ml. Good mixing can occur as long as the volume of the mixing chamber is greater than twice this volume.

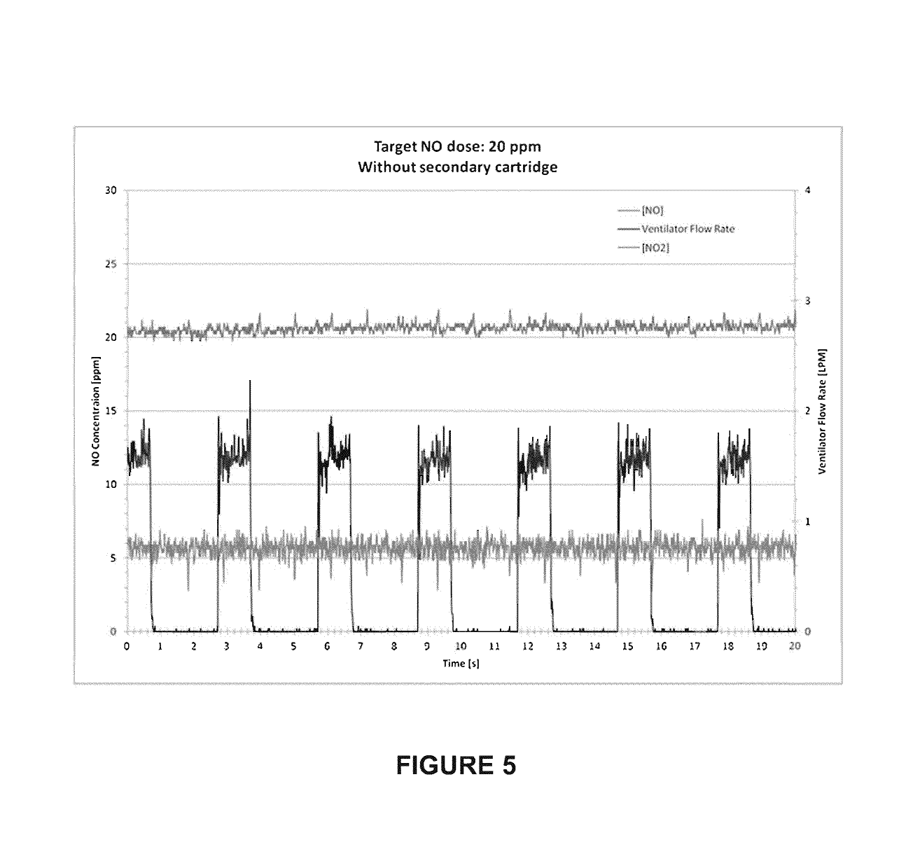

On the other hand, FIG. 5 shows the same response but without the receptacle, shown as the mixing receptacle in FIG. 3, in line with the patient. The NO₂ levels read around 0.6 ppm, which would be unacceptable for a neonate. The receptacle converts all of the NO₂ that was formed back into NO. These two figures clearly demonstrate the effect of a receptacle for converting NO₂ into NO, namely the receptacle reduced the NO₂ level as measured at the patient from 0.6 to 0 ppm.

The mixing performance of the receptacle was assessed using a high speed chemiluminescence detector with a 90% rise time of 250 msec. A very high speed NO detector was needed to catch the intra-breath variability of nitric oxide.

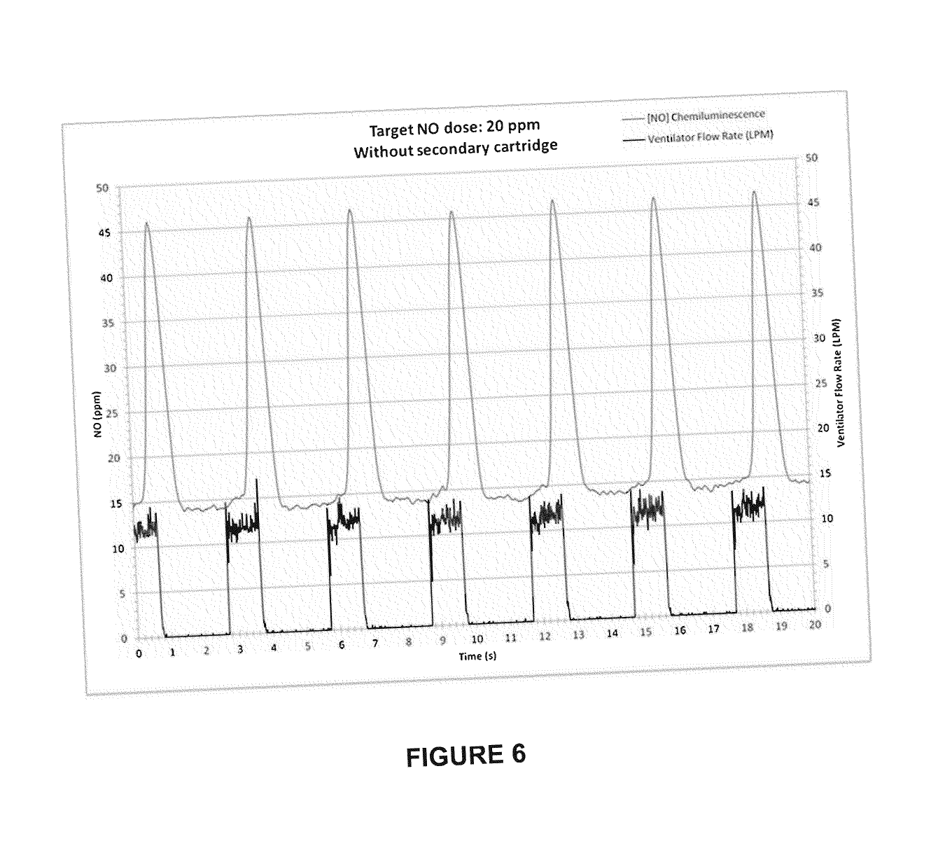

FIG. 6 shows the response of the system without the receptacle for mixing the gases (no mixing function). This chart shows the high speed version of the NO waveform presented in FIG. 5. The bottom line shows the flow rate of the ventilator. As can be seen, the absence of the receptacle introduced spikes of 30 ppm of nitric oxide (top) during the inspiratory time. Intra-breath variability of this magnitude is unacceptable.

Previous technology partially solved this problem by tracking the rapid intra-breath flow changes in the ventilator circuit and uses the electronic signal from the flow sensor to synchronize the valve that introduces the NO into the circuit. This is a difficult and complex electronic solution that requires high speed sensors and very fast computer algorithms operating in real time. Because it is so difficult to execute, the FDA (in their Guidance document) allows the NO to vary from 0 to 150% of the mean, if the total duration of these transient concentrations did not exceed 10% of the volumetric duration of the breath.

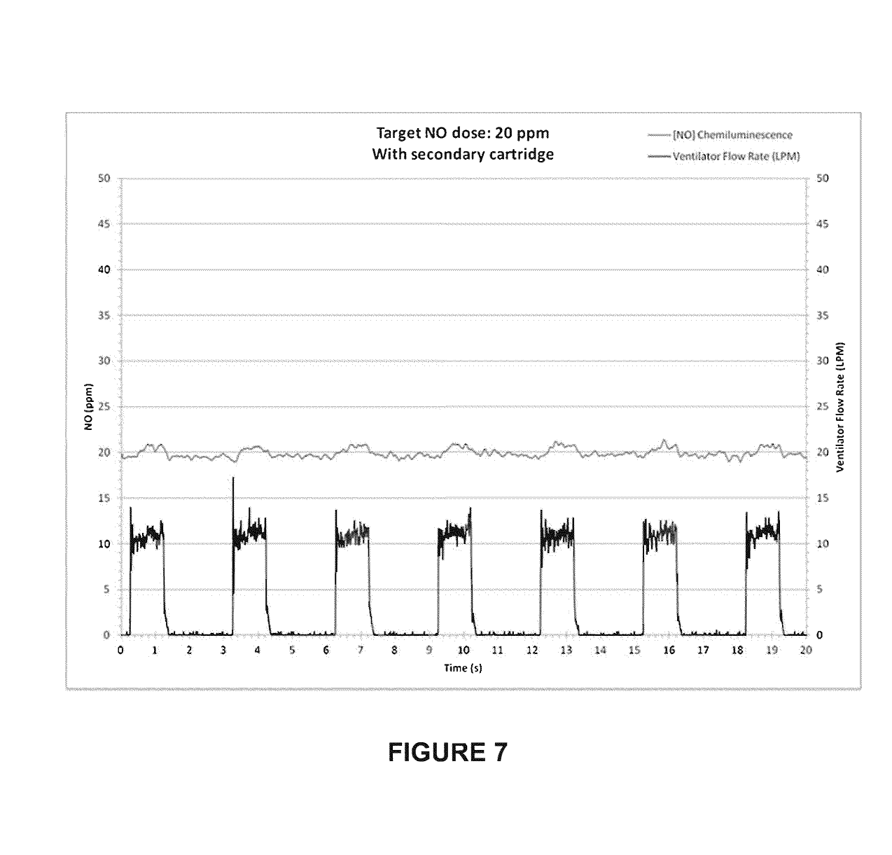

FIG. 7 shows the high speed NO version of FIG. 4 including a receptacle. The high speed detector was able to detect intra-breath variations as low as 1 ppm for the same ventilator settings used in FIG. 6. (In FIG. 4, the pulsations are not shown on the NO reading since the time response of the electro-chemical cell and associated electronics was significantly greater than the time between breaths.) The only difference was the addition of the receptacle which provides the mixing function.

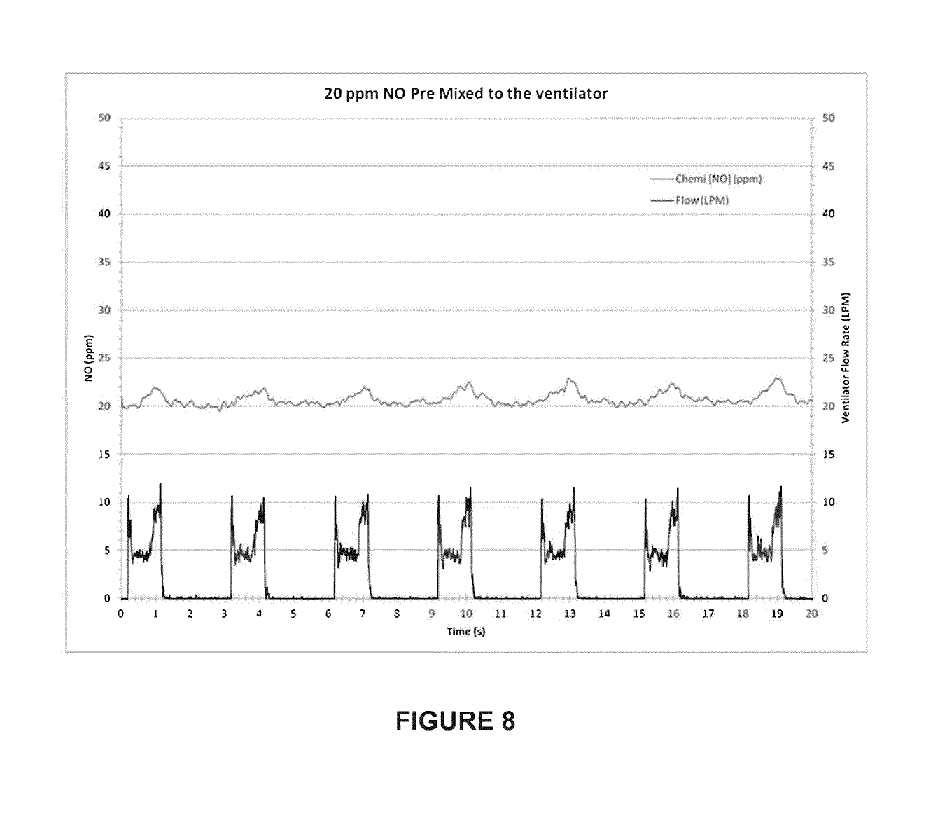

Ideal mixing can happen when the NO gas is premixed and delivered directly using the ventilator. This perfect mixing condition can provide a baseline in order to validate chemiluminescence measurements under pulsing conditions. A blender was used to premix 800 ppm of NO with air to generate a 20 ppm gas to be delivered using a ventilator only. Chemiluminescence was used to measure the NO delivered to the artificial lung. FIG. 8 shows the results. From the peaks in the NO plot (top), it is evident that the chemiluminescence device was affected by the pulsing nature of the flow (bottom). The NO measurements were almost flat but some variations were still present.

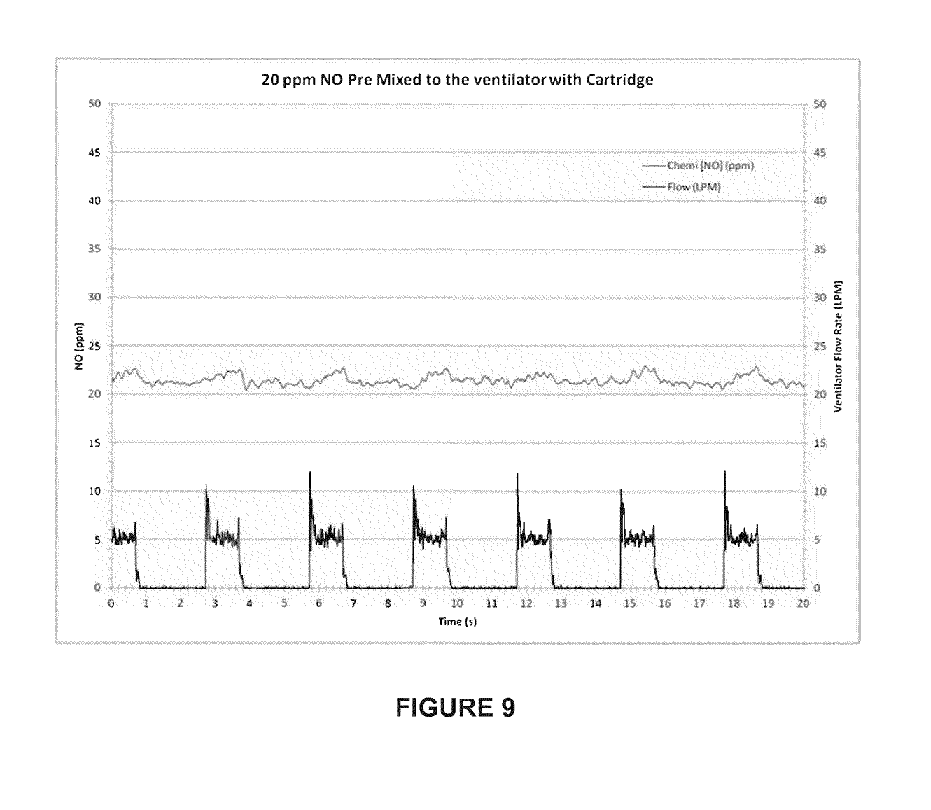

FIG. 9 shows the same experiment but the system includes a receptacle within the breathing circuit. The small amplitude oscillations were present in the NO measurements (top). From these simple experiments, it was concluded that the pulsing flow from the ventilator can provide a perfectly flat NO response using the chemiluminescence device. Furthermore, these oscillations may be due to the pressure changes in the breathing circuit since they were synchronized with the ventilator flow rate measurements (bottom). The intra breath variation that was achieved by mixing in the cartridge was indistinguishable from ideal and what can be achieved using premixed gases. In addition, the NO₂ impurity level is reduced to almost 0.0 ppm.

Constant NO injection into the breathing circuit can be a simple and viable technique as long as a receptacle is both a mixer with sufficient volume and can remove NO₂ from the circuit or can convert the NO₂ back into NO.

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

What is claimed:

1. A method of delivering nitric oxide, comprising:
mixing a first gas including oxygen and a second gas including a nitric oxide-releasing agent within a receptacle to form a gas mixture, wherein the receptacle includes an inlet, an outlet and a reducing agent;
contacting the nitric oxide-releasing agent in the gas mixture with the reducing agent to generate nitric oxide; and
delivering the gas mixture including nitric oxide from the receptacle to a mammal.

2. The method of claim 1, wherein the nitric oxide-releasing agent is nitrogen dioxide.

3. The method of claim 1, wherein the first gas is air.

4. The method of claim 1, wherein the first gas is oxygen-enriched air.

5. The method of claim 1, wherein the second gas includes an inert gas.

6. The method of claim 1, wherein the second gas includes oxygen.

7. The method of claim 1, wherein the second gas includes air.

8. The method of claim 1, wherein the concentration of nitric oxide in the gas mixture delivered is at least 0.01 ppm and at most 40 ppm.

9. The method of claim 1, wherein the concentration of nitric oxide in the gas mixture delivered is at least 0.01 ppm and at most 2 ppm.

10. The method of claim 1, wherein the mammal is a human.

11. The method of claim 1, wherein delivering the gas mixture including nitric oxide from the receptacle to the mammal includes passing the gas mixture through a delivery conduit located between the receptacle and a patient interface.

12. The method of claim 11, wherein the volume of the receptacle is greater than the volume of the delivery conduit.

13. The method of claim 12, wherein the volume of the receptacle is at least two times the volume of the delivery conduit.

14. The method of claim 12, wherein delivering the gas mixture including nitric oxide from the receptacle to the mammal includes intermittently providing the gas mixture to the mammal.

15. The method of claim 12, wherein delivering the gas mixture including nitric oxide from the receptacle to the mammal includes pulsing the gas mixture.

16. The method of claim 15, wherein pulsing includes providing the gas mixture for one or more pulses of 1 to 6 seconds.

17. The method of claim 16, wherein delivering the gas mixture including nitric oxide from the receptacle to the mammal includes providing the gas mixture to the mammal in a pre-determined delivery sequence of one or more pulses.

18. The method of claim 15, wherein the volume of the receptacle is greater than the volume of the gas mixture in a pulse.

19. The method of claim 18, wherein the volume of the receptacle is at least twice the volume of the gas mixture in a pulse.

20. The method of claim 15, wherein the gas mixture is stored in the receptacle between pulses.

21. The method of claim 1, comprising storing the gas mixture in the receptacle for a predetermined period of time.

22. The method of claim 21, wherein the predetermined period is at least 1 second.

23. The method of claim 15, wherein pulsing includes providing the gas mixture for two or more pulses and the concentration of nitric oxide in each pulse varies by less than 10%.

24. The method of claim 15, wherein pulsing includes providing the gas mixture for two or more pulses and the concentration of nitric oxide in each pulse varies by less than 10 ppm.

25. The method of claim 1, wherein the concentration of nitrogen dioxide in the gas mixture delivered to the mammal is less than 0.5 ppm.

26. The method of claim 1, comprising communicating the first gas through a gas conduit to the receptacle.

27. The method of claim 26, comprising supplying the second gas into the gas conduit immediately prior to the receptacle.

28. The method of claim 26, comprising supplying the second gas at the receptacle.
